(19) United States
(12) Patent Application Publication
(10) Pub. No.: US 2019/0021338 A1
(43) Pub. Date: Jan. 24, 2019

(54) Peptides and rhamnolipid liposomes inhibit bacterial replication in plants, bushes and trees.

(71) Applicant: Keith DeSanto, St Petersburg, FL (US)

(72) Inventor: Keith DeSanto, St Petersburg, FL (US)

(21) Appl. No.: 15/946,277

(22) Filed: Apr. 5, 2018

Related U.S. Application Data
(63) Continuation-in-part of application No. 62/517,264, filed on Jun. 9, 2017.

(51) Int. Cl. A01N 63/02; C07K 14/00

(52) U.S. Cl. CPC A01N 63/02; C07K 14/00

(57) ABSTRACT

Peptides and rhamnolipid liposomes inhibit bacterial replication in plants, bushes and trees. This invention is about internalization of a peptide inside a rhamnolipid liposome. In this invention, the chemically synthesized peptide was ParE3, an analogue from ParE protein that acts on a Toxin-Antitoxin system. This peptide is able to inhibit DNA Gyrase and Topoisomerase IV (Topo IV) activities, blocking the DNA bacterial replication, regulating its cell growth for new antimicrobial agricultural applications.

Because Rhamnolipid has both hydrophilic and hydrophobic components, Rhamnolipid facilitates its entry into cell membranes by breaking down the cell wall of bacteria. Bacteria Cell permeability is created by using this application.

This invention is about internalization of a peptide inside a rhamnolipid liposome. In this invention, the chemically synthesized peptide was ParE3, an analogue from ParE protein that acts on a Toxin-Antitoxin system. This peptide is able to inhibit DNA Gyrase and Topoisomerase IV (Topo IV) activities, blocking the DNA bacterial replication, regulating its cell growth for new antimicrobial drugs. The Rhamnolipid Peptide Liposomes antimicrobial activity is impaired by its difficult bacterial cell membrane permeability.

The biotechnological potential application of peptides analogues from ParE into liposomes would be able to increase its bioavailability. Rhamnolipid is used for producing liposomes because of its low toxicity, high biodegradability and improved antimicrobial activities. As a result, the ParE3 peptide internalization into rhamnolipid liposomes increased cell permeability and bioavailability, consequently microbial inhibition was obtained.

This invention is about internalization of a peptide inside a rhamnolipid liposome. In this case, the chemically synthesized peptide was ParE3, an analogue from ParE protein that acts on Toxin-Antitoxin system. This peptide is able to inhibit DNA Gyrase and Topoisomerase IV (Topo IV) activities, blocking the DNA bacterial replication, regulating its cell growth, which is an interesting mechanism for the development of new antimicrobial drugs; by the way, its antimicrobial activity is impaired by its difficult bacterial cell membrane permeability.

Considering the biotechnological potential application of peptides analogues from ParE, we started thinking that its internalization into liposomes would be able to increase its bioavailability. We chose rhamnolipids for producing liposomes because of its low toxicity, high biodegradability and comproved antimicrobial activity. As a result we already have that ParE3 peptide internalization into rhamnolipid liposomes was successful, increased cell permeability and bioavailability, consequently better results for microbial inhibition were obtained.

Rhamnolipids are one of the most important biosurfactant types (Haba et al., 2013) and are mainly produced by the fermentation rote of Pseudomonas aeruginosa, but they also can be produced by Rhodotorula taiwanensis, Lactobacillus Plantarum, Pseudomonas Rhizophila, Pseudomonas Chlororaphis and Burkholderia sp. They are recognized as a “green production” due to their low environmental cytotoxicity, but they also have high emulsification potential and antimicrobial activities. The two components of Rhamnolipid consist of a hydrophilic (water attracting) part and a hydrophobic (water hating) part. Because rhamnolipid is amphipathic (having both hydrophilic and hydrophobic parts), this characteristic makes it easy to penetrate cell membranes of bacteria that cause disease.

### Rhamnolipid Production

The production medium consisted of a Ca-free mineral salt solution with 15.0 g/L NaNO3, 0.5 g/L MgSO4×7 H2O, 1.0 g/L KCl and as a phosphate source 0.3 g/L K2HPO4. As sole carbon source soybean oil with a starting concentration of 250 g/L was used and 1 mL/L of the above-mentioned trace element solution was added.

The trace element solution contained 2.0 g/L sodium citrate×2 H2O, 0.28 g/L FeCl3×6 H2O, 1.4 g/L ZnSO×7 H2O, 1.2 g/L CoCl2×6 H2O, 1.2 g/L CuSO4×5 H2O, and 0.8 g/L MnSO4×H2O. The fermentation was carried out at 37° C., pH 6.9, and the process was carried out for 158 h. The rhamnolipid produced was purified by acidification and then an extraction was carried out using ethyl acetate.

The molecular weight of the rhamnolipid is between 475 g/mol and 677 g/mol.

Because Rhamnolipid has both hydrophilic and hydrophobic components, Rhamnolipid facilitates its entry into cell membranes by breaking down the cell wall of bacteria causing diseases in plant, bushes and trees. Mixing the rhamnolipid with liposomes, increases the effect rate.

1. Applying a peptide inside a rhamnolipid liposome to plants, trees and bushes to kill diseases and plant pathogens affecting those plants, trees and bushes.

2. spraying a peptide inside a rhamnolipid liposome on a plant, bush or tree to kill diseases and plant pathogens affecting those plants, trees and bushes.

3. Including a peptide inside a rhamnolipid liposome with a fertilizer to kill diseases and plant pathogens affecting those plants, trees and bushes.

4. Applying a peptide inside a rhamnolipid liposome to plants, trees and bushes to prevent plant pathogens from affecting those plants, trees and bushes.

5. Spraying a peptide inside a rhamnolipid liposome on a plant, bush or tree to prevent diseases and plant pathogens affecting those plants, trees and bushes.

6. Including a peptide inside a rhamnolipid liposome with a fertilizer to prevent diseases from affecting plant pathogens affecting those plants, trees and bushes.
